(19) United States
(12) Patent Application Publication
(10) Pub. No.: US 2026/0137871 A1
(43) Pub. Date: May 21, 2026

(54) ACTIVE AGENT AMPOULE FOR A MEDICAL SYRINGE, AND METHOD FOR FILLING SUCH AN ACTIVE AGENT AMPOULE

(71) Applicant: INDUCTIO AG, Solothurn (CH)

(72) Inventor: Horst KOLLER, Solothurn (CH)

(73) Assignee: INDUCTIO AG, Solothurn (CH)

(21) Appl. No.: 18/867,787

(22) PCT Filed: May 11, 2023
(86) PCT No.: PCT/EP2023/062626

§ 371 Date: Jan. 23, 2026

(30) Foreign Application Priority Data
May 20, 2022 (EP) 22174670.4

(51) Int. Cl. A61M 5/28; A61M 5/24; A61M 5/32

(52) U.S. Cl. CPC A61M 5/288; A61M 5/2466; A61M 5/281; A61M 5/3202; A61M 2005/2488; A61M 2005/3206; A61M 2205/0216; A61M 2205/19; A61M 2207/00

(57) ABSTRACT

An active ingredient cartridge (2) for a medical syringe (1) with an essentially cylindrical hollow body (16) made of a first plastic material that can be filled with an active ingredient is intended to facilitate filling of the active ingredient and also to enable high throughput rates during filling in automated processes. For this purpose, according to the invention, the hollow body (16) is provided at one end (4) with an integrated closure body (50) made of a second plastic material which is softer than the first, wherein the hybrid body formed by the hollow body (16) and the closure body (50) is obtained by a 2K injection molding process.

### DESCRIPTION

The invention relates to a cartridge for an active ingredient for a medical syringe, in particular for use in local anesthesia, for example in dental treatments. It further relates to a method for filling such an active ingredient cartridge with active ingredient.

Medical syringes with cartridges prefilled with the intended active ingredient are used in large numbers in a wide range of applications, in particular for local anesthesia in dental treatment. In such a syringe, as known for example from EP 3 738 629 A1, the prefilled ampoule of active ingredient, also known as a cartridge, is provided on the one hand with a handle or frame and on the other hand with a needle head or needle carrier, so that the syringe is ready for use. The needle head or needle carrier comprises the actual syringe needle, the proximal end of which pierces the end seal of the cartridge when the needle head is attached to the cartridge and is thus connected to the interior of the cartridge on the media side. The handle, on the other hand, usually comprises an actuating plunger that can be displaced in its longitudinal direction for the syringe plunger or stopper arranged in the cartridge, via which the active ingredient can be pressed in the direction of the needle and dispensed via this.

The use of such prefilled active ingredient cartridges makes it much easier for the user, e.g. the dentist, to handle them, as the user does not need to fill the active ingredient in a comparatively time-consuming manner. In addition, such active ingredient cartridges can be filled centrally by machine and automatically in large quantities and at high cycle rates. Nevertheless, there is a need to make the handling of such systems and the filling process itself even simpler and therefore more efficient.

The invention is now based on the object of specifying a cartridge for an active ingredient for a syringe of the above-mentioned type, with which this need is met. Furthermore, a particularly favorable method for filling such an active ingredient cartridge shall be specified.

With regard to the active ingredient cartridge, this task is solved according to the invention with a substantially cylindrical hollow body made of a first plastic material which can be filled with an active ingredient and which is provided at one end with an integrated closure body made of a second plastic material which is softer than the first, obtained by a 2-component injection molding process.

The invention is based on the consideration that the number of components or parts used and also the work steps for assembling the cartridge should be kept as low as possible for particularly efficient and resource-saving manufacture and also filling of the cartridge. To make this possible, it is planned to manufacture the cylindrical hollow body together with the closure body from the outset and in a single work step. A 2K injection molding process is provided for this purpose, whereby the “2K hybrid body” produced in this way can also be fed to the filling system immediately after its production.

For functional reasons, the plastics for the hollow body on the one hand side and the closure body on the other are advantageously selected appropriately. The hollow body or cartridge body can be made as a plastic part from cyclo-olefin copolymer (COC), PC or PP, but in a particularly preferred embodiment is made as a plastic part from cyclo-olefin polymer (COP). These materials are characterized by their high breaking strength and glass-like transparency. They do not release any alkali ions, so there is no risk of a pH shift in the active ingredient. According to the invention, this material is therefore regarded suitable and used even for the primary packaging of even the most demanding medical products, especially for sensitive biotechnologically produced active ingredients. In addition, this material is particularly suitable for the intended production by injection molding and thus for particularly precise dimensioning.

In contrast, the closure body should offer the possibility that a syringe needle can be easily pierced through it to remove the active ingredient, i.e. to administer the active ingredient when the syringe is actuated. For this purpose, the closure body advantageously forms a membrane in a frontal central area that can be pierced by a hollow needle. In an advantageous further development, a fixing skirt is formed around this membrane, which lies against the inner wall of the hollow body in a sealing manner.

The production of the hybrid body of the hollow body of the active ingredient cartridge with integrated closure body in a single work step in a 2K injection molding process, as provided for in one aspect of the invention, can also be used dedicatedly in a particularly advantage manner to fix the position of the closure body in the hollow body by means of form closure or undercuts. For this purpose, the hollow body advantageously has a circumferential bead on the inside in its end area containing the closure body, which engages in a corresponding circumferential outer groove in the fixing skirt.

According to one aspect of the invention, the active substance cartridge of the type described is part of a medical syringe, wherein a needle head carrying a hollow needle and a handle can be attached to the end of the active substance cartridge.

In one embodiment, which is regarded as independent and independently inventive, a medical syringe, in particular of the type described above, is provided with a needle guard. In order to avoid or reduce the risk of contaminations or injury after the use of medical syringes and in particular to avoid the multiple use of syringes needles by different users, syringes with needle protection systems are increasingly being used. Such needle protection can, for example, be designed in the form of a pull back or retraction system, in which the syringe needle is retracted into the syringe body and completely enclosed by it after dispensing the active ingredient contained in the syringe. Access to the syringe and thus a risk of injury, or even the risk of multiple use of the same needle, can thus be largely excluded.

In the present case, such a needle guard is realized in an embodiment which is regarded as independently inventive, in that the needle head has a base body to which a protective body is pivotably attached via a joint to form a needle guard sleeve, the length of which exceeds the free length of the hollow needle and thus projects beyond it at the end.

According to one aspect of the invention, the cartridge for the active ingredient of the type described above is manufactured by producing the hollow body and the closure body provided therein in a joint work step in a 2-component injection molding process.

With regard to the method for filling an active ingredient cartridge of the type described above with active ingredient, the mentioned object is achieved in accordance with one aspect of the invention by bringing the hollow body with its longitudinal direction into a vertical position, with the end provided with the closure body being aligned in downwards direction and the hollow body then being filled via its open end. A suitable syringe plug can then be inserted into this open end so that the filled active ingredient is sealed tightly in the hollow body.

An embodiment of the invention is explained in more detail with reference to a drawing. it shows:

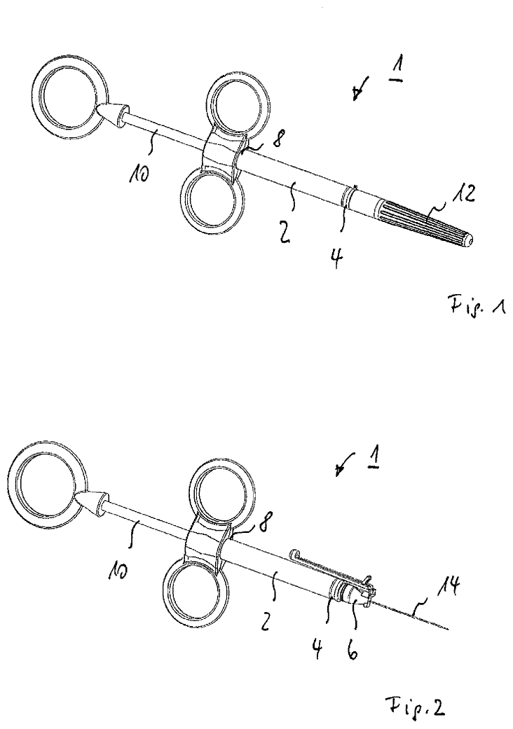

FIG. 1 a perspective view of a medical syringe, in particular for use in local anesthesia, for example during dental treatment,

FIG. 2 a perspective view of the syringe according to FIG. 1 with the needle protection cap removed,

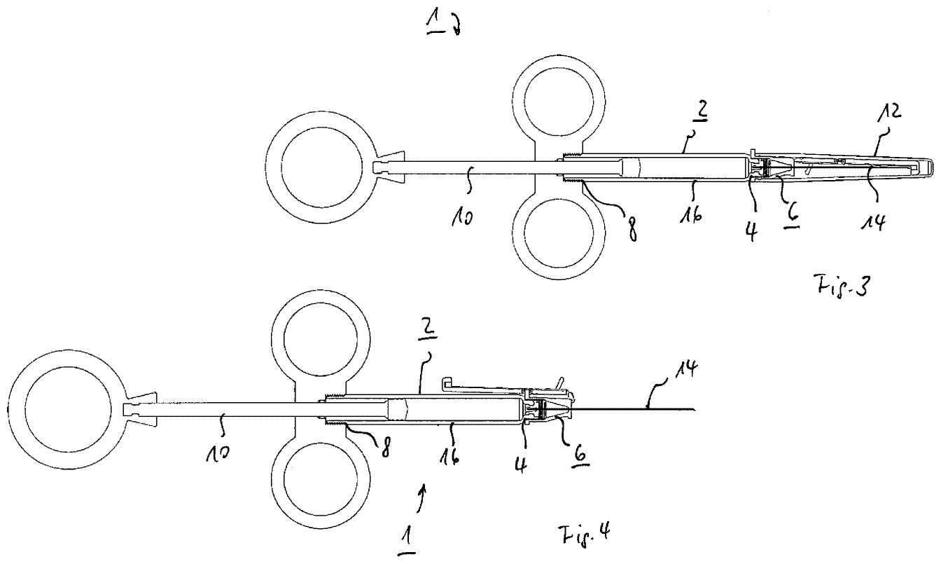

FIG. 3 a longitudinal section of the syringe shown in FIG. 1,

FIG. 4 a longitudinal section of the syringe shown in FIG. 2,

FIG. 5 a perspective view of the needle head of the syringe according to FIG. 1,

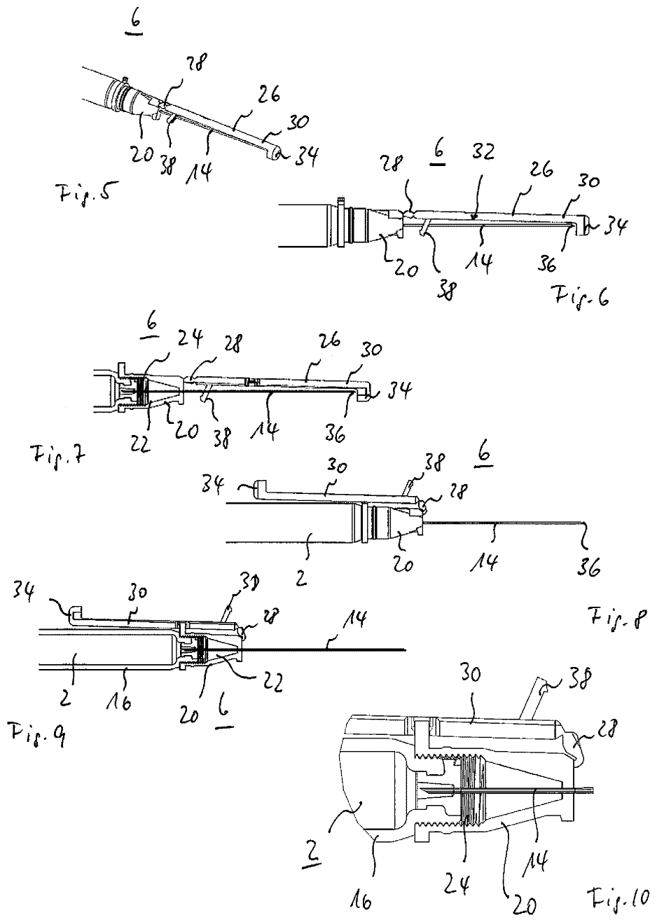

FIG. 6 a side view of the needle head of the syringe as shown in FIG. 1,

FIG. 7 the needle head of the syringe according to FIG. 1 in longitudinal section,

FIG. 8 a side view of the needle head of the syringe according to FIG. 1 in ready-to-use condition,

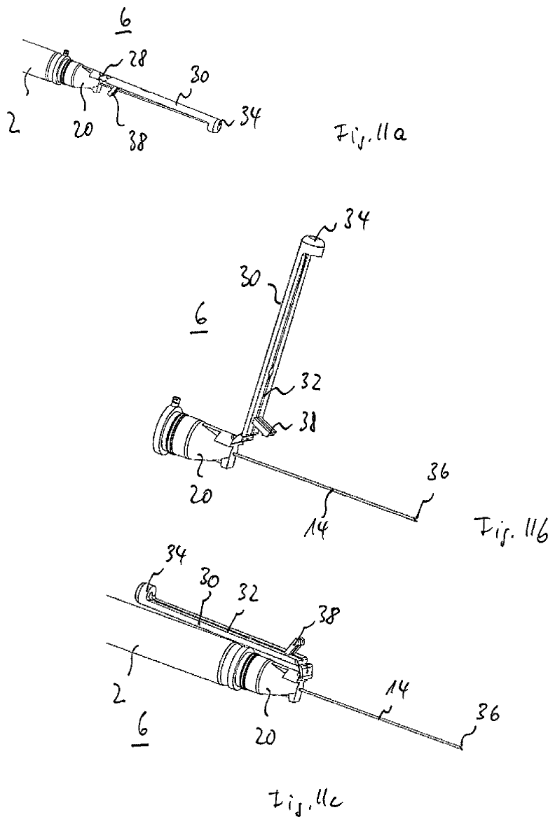

FIG. 9 the needle head as shown in FIG. 8 in longitudinal section,

FIG. 10 a sectional enlargement of FIG. 9,

FIG. 11 a sequence when a protective body is swung away from the syringe needle,

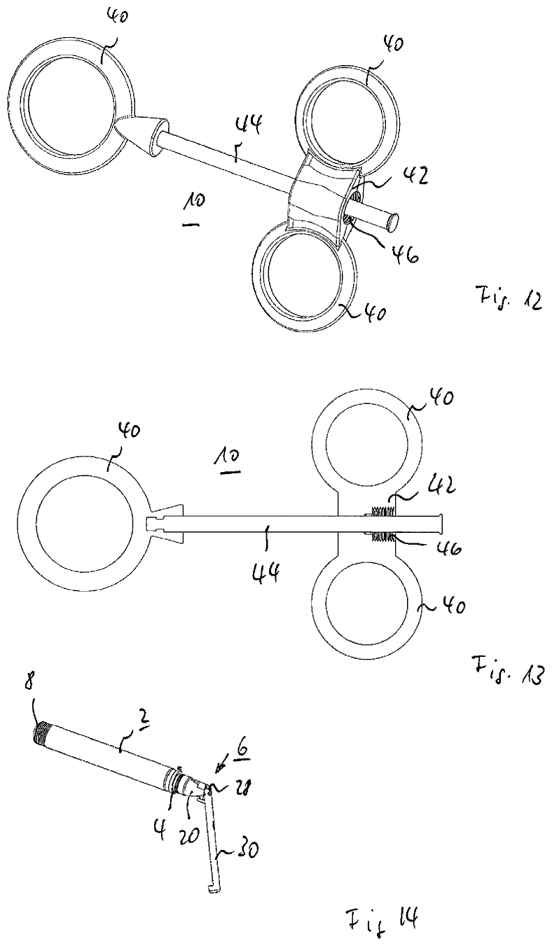

FIG. 12 a perspective view of a handle of the syringe as shown in FIG. 1,

FIG. 13 the handle as shown in FIG. 12 in longitudinal section,

FIG. 14 an ensemble of empty active substance cartridge and needle head with kinked needle intended for disposal,

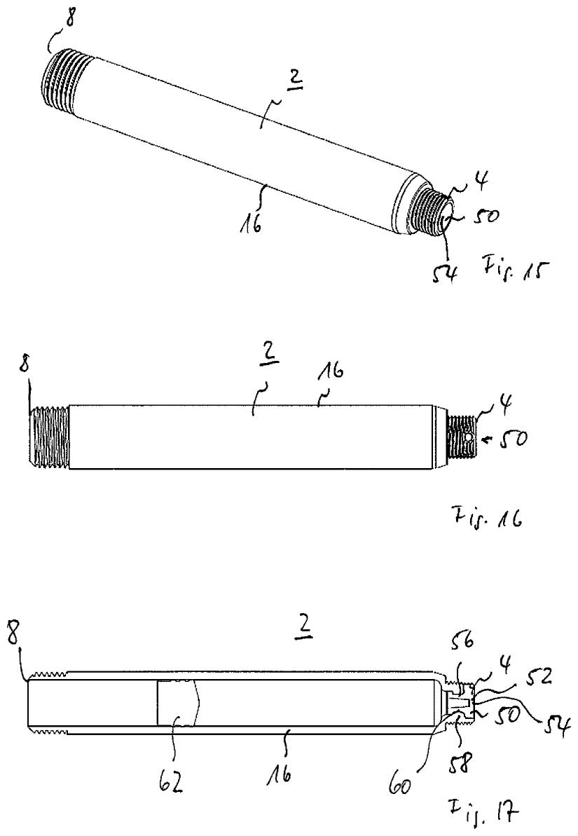

FIG. 15 a perspective view of the active ingredient cartridge of the syringe according to FIG. 1,

FIG. 16 the active ingredient cartridge according to FIG. 15 in lateral view, and

FIG. 17 the active ingredient cartridge according to FIG. 15 in longitudinal section.

Identical parts are marked with the same reference signs in all figures.

The medical syringe 1 according to FIG. 1 essentially comprises three components, namely an active ingredient cartridge 2 (also known as a cartridge unit), which is connected at its distal end 4 to a needle head 6 or needle holder and at its proximal end 8 to a handle 10. In FIG. 1, the syringe 1 is also shown with a needle protection cap 12 attached to the needle head 6. FIG. 2, on the other hand, shows the syringe 1 in an operational state with the needle protection cap 12 removed, so that the hollow needle 14 attached to the needle head 6 is exposed. FIGS. 3 and 4 show the syringe 1 as shown in FIG. 1 and FIG. 2 respectively in longitudinal section.

The active ingredient cartridge 2 comprises a hollow body 16, which is cylindrical or tubular according to a conventional design and forms a syringe housing and is intended to hold the medically active ingredient. The active substance cartridge 2 is designed as a prefilled active substance cartridge, which is filled with the intended active substance in large quantities at a central manufacturing location and then delivered to the user, for example the dentist. In order to make the syringe 1 ready for use, the user must attach the needle head 6 on the one hand and the handle 10 on the other to the hollow body 16.

The needle head 6 or needle holder can be screwed onto the front or distal end 4 of the hollow body 16 or can also be plugged on, depending on which design is selected for the connection. The needle head 6, which is still provided with the needle protection cap 12 at this moment of attachment, has a design that is considered to be inventive in its own right. This takes account of the fact that there is an increasing need, and in some cases this has already been incorporated into legal or regulatory provisions, to equip medical syringes with so-called needle protection systems. Increasing legal requirements for a safe working environment in various countries oblige employers in the medical sector, for example, to protect their employees from needlestick injuries. To this end, a system of protective caps can be provided which are placed over the needle when it is not in use, or so-called retraction systems can be provided in which the needle is retracted after use into the syringe housing, which has been emptied of the active ingredient in the meantime, so that the needle tip is no longer exposed.

In the present embodiment, which is regarded as independently inventive, the needle head 6 or needle holder that can be attached to the hollow body 16 is provided with an integrated needle protection system. The needle head 6—after removal of the needle protection cap 12—is shown enlarged in perspective view in FIG. 5, in lateral view in FIG. 6 and in longitudinal section in FIG. 7.

As can be clearly seen from these illustrations, the needle head 6 comprises a base body 20 forming the actual needle holder, preferably made of a hard plastic such as PP, POM or PC. The base body 20 supports and carries the hollow needle 14; it is provided on the inside with a receiving channel 22 into which the distal end 4 of the hollow body 16 can be inserted. Shown here is the variant in which the needle head 6 is to be screwed onto the distal end 4 of the hollow body 16, and accordingly, in the embodiment example shown, the base body 20 has an internal thread 24, preferably a fine internal thread, in its receiving channel 22.

To provide the desired needle protection, a needle protection cover 26 is pivotably attached to the base body 20 via a joint 28. The needle protective cover 26 is formed by an elongated protective body 30, the length of which exceeds the free length of the hollow needle 14 and thus projects beyond it at the end. Furthermore, the width of the protective body 30 is selected in such a way that it clearly projects outwards beyond the hollow needle 14 on both sides. The protective body 30 can also have a receiving channel 32 on the inside, in which the hollow needle 14 can be embedded in the position of the protective body 30 shown in FIGS. 5-7 . A cover plate 34 is formed on the end of the protective body 30, which covers the free end 36 of the hollow needle 14 in the position shown there. In the position shown in FIGS. 5-7 , the free end 36 of the hollow needle 14 is thus completely shielded, and no contact and thus no injury by the hollow needle 14 is possible.

The system is in this position when the outer protective cap 12 is removed. As the hollow needle 14 is still shielded by the protective body 30 in this state, it is not yet possible to pierce the needle 14. In order to make it insertable, an actuating element 38 formed on the protective body 30 is therefore pivoted away from the hollow needle 14 around the joint 28 so that it is now exposed and released. In this state, in which the syringe 1 is now ready to dispense the active ingredient, the needle head 6 is shown in side view in FIG. 8, enlarged in longitudinal section in FIG. 9 and in longitudinal section in FIG. 10.

The swinging away of the protective body 30 from the hollow needle 14, which exposes it and thus makes it available for use, is shown again in the sequence according to FIG. 11 for clarification, starting from the starting position directly after removal of the outer needle protection cap 12 (FIG. 11a) via an intermediate position with the protective body 30 half swung away (FIG. 11b) to the end position (FIG. 11c), in which the protective body 30 is completely swung away from the needle 14 and rests against the back of the outside of the hollow body 16.

After use of the syringe 1, the protective body 30 can be pivoted back around the joint 28 so that it again shields the hollow needle 14 as described above and thus injuries caused by touching the needle 14 are safely avoided. However, in order to further reduce the risk of injury to the needle 14, which is then used, a further aspect of the invention provides for the protective body 30 to be pivoted even further around the joint 28 until the needle 14 breaks off in its needle bearing in the base body 20. This finally fixes the needle 14 in its position within the shielding area of the protective body 30, and it also makes it clear that the syringe 1 has already been used and that the (now used) active ingredient cartridge 2 is to be disposed of together with the now broken needle 14 that is still attached to it. This reliably prevents errors and mix-ups that could, for example, lead to the syringe 1 being accidentally reused.

The aforementioned components of the needle guard 26 can be attached to the hollow needle 14 in the form of an “overmolding” in a single, joint working step.

As described above, the handle 10 must also first be attached to the hollow body 16 before the syringe 1 is used. As can be seen from the perspective view in FIG. 12 and the longitudinal sectional view in FIG. 13, it comprises a base element 42 provided with finger rings 40, in which an actuating plunger 44, also provided with a finger tab 40 at the end, is guided displaceably in its longitudinal direction. A screw connection is also provided here in the embodiment example for connection to the active ingredient cartridge 2, and accordingly the base element 42 is provided with a thread 46 in which the proximal end 8 of the active ingredient cartridge 2 can be screwed into. With regard to the usual design objectives of a construction that is simple in itself but of high quality and in particular meets high safety requirements, the aforementioned components of the handle piece 10 are made of polyphenylsulfone (PPSU) in the embodiment example, a highly technical special plastic that can be sterilized almost as often as required with high mechanical stability and load-bearing capacity and is considered a metal substitute for medical products. In its entirety, the handle 10 can therefore be sterilized many times and repeatedly, particularly due to its choice of material, and can therefore be reused many times.

After the active ingredient has been dispensed in the manner described above, the—now empty—active ingredient cartridge 2 can be separated from the handle piece 10 again by loosening the screw connection between the hollow body 16 and the handle piece 10. Together with the needle head 6 and the hollow needle 14 attached to it, which has in the meantime been bent together with the protective body 30, the ensemble of needle head 6 and—emptied—active ingredient cartridge 2 can be disposed of. This ensemble is shown in perspective view in FIG. 14.

The design and layout described above offer the following advantages for medical staff and patients in particular:
Very easy handling
Extremely low weight
Controlled system activation with just one hand
irreversible and clearly recognizable snap-in effect
unchanged injection technique
Normal disposal and low waste; the needle protection concept means it is not hazardous waste
No additional space required
Needle and syringe contents are completely visible

According to a further aspect of the invention, which is regarded as independent and independently inventive, the active ingredient cartridge 2 is specifically designed for a highly efficient manufacturing and filling process suitable for large quantities. For this purpose, the active ingredient cartridge 2, which is shown in perspective view in FIG. 15, in side view in FIG. 16 and in longitudinal section in FIG. 17, is designed as a hollow body 16, which is provided at its distal end 4, intended for connection to the needle head 6, with an integrated closure body 50 that can be pierced by the proximal end of the hollow needle 14. The closure body 50 is designed as a three-dimensional molded body, as can be seen in particular in the longitudinal sectional view in FIG. 17. In a frontal central area 52, this forms a comparatively thin membrane 54 made of TPE, which can be pierced by the proximal end of the hollow needle 14 when the needle head 6 is attached.

A fixing skirt 56 is formed around the diaphragm 54, which lies tightly against the inside wall of the hollow body 16 and fixes the closure body 50 in its position in the hollow body 16. In order to reliably prevent unintentional displacement of the closure body 50 relative to the hollow body, for example slipping out, particularly in the longitudinal direction of the system, the hollow body 16 has a circumferential bead 58 on the inside in its distal end region, which engages in a corresponding circumferential outer groove 60 in the fixing skirt 56. This fixes the position of the closure body 50 in the hollow body 16 in the longitudinal direction.

With regard to the choice of material for the syringe housing or the hollow body 16 forming it, which is considered to be inventive in its own right, particular account is taken of high demands on the reliable temporary storage of the medical active ingredient together with a particularly high level of safety when handling the components. The syringe barrel 16, which is designed as a cylindrical hollow body, can be made of COC, PC or PP, but in the embodiment shown it is made of the high-performance plastic cyclo-olefin polymer (COP), in an embodiment considered to be inventive. This material is characterized by its high breaking strength and glass-like transparency. It also does not release alkali ions, so there is no risk of a pH shift in the active ingredient stored.

In contrast, the closure body 50 is made of the comparatively soft TPE, so that the intended piercing of the hollow needle 14 is also possible without any problems while maintaining a high level of tightness.

In an embodiment that is also regarded as independently inventive, a 2-component injection molding process is provided for the manufacture of the hollow body 16 provided with the closure body 50. The components are manufactured together in a single process, so that the intended positioning of the closure body 50 in the hollow body 16 with less displacement can be achieved easily and simply. The syringe body 6 is preferably manufactured by injection molding, whereby, among other things, the shape is produced in such a way that possible dead volumes in the interior are kept particularly low.

Corresponding to the closure body 50, a plug 62, also made of TPE, is provided which can be moved inside the hollow body 16. This is dimensioned in such a way that it lies tightly against the inner wall of the hollow body 16; the reservoir for the active ingredient is thus formed in the longitudinal area of the interior of the hollow body 16 between the closure body 50 and the stopper 62, in which the active ingredient can be held. When the needle head 6 is attached, with the hollow needle 14 projecting through the membrane 54 into this reservoir volume, the stopper 62 can be moved towards the distal end 4 and thus towards the closure body 50 inside the hollow body 16, so that the active ingredient is pressed out of the interior by the hollow needle 14. When the syringe 1 is used and the handle 10 is mounted, the actuating plunger 44 strikes the stopper 62 with its free end so that it can be displaced by means of the actuating plunger.

In the embodiment shown, the active ingredient cartridge 2 is designed for a screw connection both with the needle head 6 and with the handle 10 and is therefore provided with a corresponding screw thread at the distal and proximal ends 4, 8. Alternatively, however, one or both of these connections could also be designed as a press or plug connection and accordingly without a thread.

According to one aspect of the invention, the outer dimensions of the active ingredient cartridge 2 are identical to those of the glass cartridges used to date. This ensures backward compatibility with existing systems and combinability with existing components such as handles.

The described design of the active ingredient cartridge 2 allows for a revolutionary, novel filling process, which is also considered to be independently inventive, which enables particularly high throughput rates and thus unit numbers during filling in a particularly simple way. In a first step, as described above, the hollow body 16 sealed at the end with the closure body 50 is produced as a 2K or hybrid component. Further on, the stopper 62 is provided. According to one aspect of the invention, the hybrid body comprising hollow body 16 and integrated closure body 50 is produced in compliance with the specifications and regulations for primary packaging materials, so that it can be transferred directly, without detours and without further intermediate steps, from the injection molding plant to the filling plant. The intermediate steps of washing, siliconizing or sterilizing otherwise required in this context are thus no longer necessary, which means a considerable process simplification.

During filling, i.e. in the filling plant, the hollow body 16 provided with the closure body 50 is then aligned vertically, i.e. with its longitudinal axis parallel to the force of gravity, in accordance with a further aspect of the invention, in such a way that the proximal end 8, which is still open in this state, points “upwards” and the distal end 4, which is closed with the closure body 50, points “downwards”, i.e. towards the earth. The filling of the active ingredient cartridge 2 with the active ingredient can thus begin. According to one aspect of the invention, this takes place in a large-scale filling system in which a large number of cartridges can be filled in parallel and at the same time. After the intended quantity of active ingredient has been introduced into the hollow body 16, the stopper 62 is finally pushed into the proximal end 8 of the hollow body 16 and thus closes the interior filled with the active ingredient. Sealing and final inspection are then carried out in the conventional manner, and the filled cartridge 2 of active ingredient is ready for shipment.

### LIST OF REFERENCE SYMBOLS

1 medical syringe
2 active ingredient cartridge
4 distal end
6 needle head
8 proximal end
10 handle
12 needle protection cap
14 hollow needle
16 hollow body
20 base body
22 receiving channel
24 internal thread
26 needle protection cover
28 joint
30 protective body
32 receiving channel
34 cover plate
36 free end
38 operating element
40 finger ring
42 basic element
44 actuating plunger
46 internal thread
50 closure body
52 frontal central area
54 membrane
56 fixing skirt
58 bead
60 external groove
62 plug

1. An active agent ampoule for a medical syringe, comprising:
a cylindrical hollow body made from a first plastic material which can be filled with an active agent; and
a closure body integrated at a one end of the cylindrical hollow body made of a second plastic material which is softer than the first plastic material;
wherein the closure body is obtained by a 2K injection molding process, and includes:
a membrane;
a fixing skirt which is molded onto the membrane in a circumferential manner and which bears in a sealing manner against an inner wall of the hollow body; and
a circumferential bead formed on the inner wall of the hollow body in a region containing the closure body, wherein the circumferential bead engages in a corresponding circumferential outer groove on the fixing skirt.

2. The active agent ampoule of claim 1, wherein the hollow body is manufactured as a plastic part from at least one of cyclo-olefin copolymer (COC), polycarbonate (PC), polypropylene (PP), or cyclo-olefin polymer (COP).

3. The active agent ampoule of claim 1, wherein the closure body forms the membrane which can be pierced by a hollow needle in a frontal central region.

4. The active agent ampoule of claim 1, wherein the closure body is made of thermoplastic elastomer (TPE).

5. The active agent ampoule of claim 1, further comprising a stopper, made of thermoplastic elastomer, the stopper being displaceable within the hollow body.

6. The active agent ampoule of claim 1, wherein the medical syringe further comprises a needle head, configured to carry a hollow needle on one end and a handle on an other end, and wherein the needle head is attached at the one end of the cylindrical hollow body.

7. The active agent ampoule of claim 6, wherein the needle head further comprises a base body and a protective body pivotally attached to the base body via a joint;
wherein a length of the protective body exceeds a free length of the hollow needle and thus projects beyond the hollow needle; and
wherein the protective body is configured to form a protective needle sheath and is pivotably mounted in such a way that, after use of the syringe, the protective body can be pivoted via the joint until the hollow needle breaks off at a connection point between the needle and in the base body.

8. The active agent ampoule of claim 1, wherein the hollow body and the closure body were produced in a joint working step in the 2K injection molding process.

9. The active agent ampoule of claim 1, wherein, when the hollow body is oriented with its longitudinal direction in a vertical position, the one end of the closure body will be oriented below an open end of the hollow body located opposite the open end, so that the hollow body can be filled via the open end.
